(19) United States
(12) Patent Application Publication
(10) Pub. No.: US 2024/0082423 A1
(43) Pub. Date: Mar. 14, 2024

(54) NANOCONE CLUSTERS SUITABLE FOR USE AS HISTOTRIPSY AGENT

(71) Applicant: ISTANBUL MEDIPOL UNIVERSITESI TEKNOLOJI TRANSFER OFISI ANONIM SIRKETI, Beykoz, Istanbul (TR)

(72) Inventor: Yasemin YÜKSEL DURMAZ, Beykoz, Istanbul (TR)

(21) Appl. No.: 18/260,089

(22) PCT Filed: Dec. 26, 2021
(86) PCT No.: PCT/TR2021/051513

§ 371 Date: Jun. 30, 2023

(30) Foreign Application Priority Data
Dec. 31, 2020 (TR) 2020/22795

(51) Int. Cl. A61K 47/69; A61K 47/64; A61N 7/00; C08B 37/16

(52) U.S. Cl. CPC A61K 47/6951; A61K 47/64; A61N 7/00; C08B 37/0015; A61N 2007/0039

(57) ABSTRACT

The present invention relates to nanocone clusters suitable for use as a histotripsy agent, to methods used for the preparation of said clusters, and to the use of the clusters according to the present invention as histotripsy agents or in drug delivery.

### TECHNICAL FIELD

The present invention relates to nanocontainer clusters suitable for use as a histotripsy agent, to methods used for the preparation of said clusters, and to the use of the clusters according to the present invention as histotripsy agents or in drug delivery.

### STATE OF THE ART

Histotripsy is a mechanical cell ablation method that works on acoustic cavitation mechanism using microsecond-long, high-frequency ultrasound (US) pulses. These ultrasound pulses generate a bubble cloud by using gas bubbles that exist in the body in dissolved form. When these bubble clouds gain enough energy and collapse (cavitation), they induce cellular destruction/ablation in the tissue in which said clouds are located. The development of cavitation requires exceedingly high pressure.

Histotripsy is a novel method that is stipulated to be used in the destruction of tumor tissues in cancer cases as it induces damage in tissues. However, creating a gas cloud out of gas bubbles without using a histotripsy agent requires generating approximately 28 MPa-30 MPa of pressure, and this pressure is high enough to induce damage in healthy, non-tumorous tissues.

Recently, nanodroplet mediated histotripsy has been developed with the aim of addressing this issue. The method mentioned above uses a perfluorocarbon (PFC), for example, perfluoropentane encapsulated polymeric nanodroplets. When this agent penetrates into the tumor, perfluoropentane contained inside the nanoparticles serves as a core in the creation of a gas cloud (cavitation) instead of the gas bubbles inside the tissue. The method has been observed to enable reducing the pressure required for creating cavitation from 28 MPa to 7 MPa, thereby preventing healthy tissues from sustaining damage during the application.

The present method comprises complex steps for the preparation of a polymer that consists of 3 blocks as the main constituent of nanoparticles, which, in turn, requires advanced synthesis capabilities as well as expertise. Another issue in this regard is that it is not possible to determine the amount of PFC encapsulated in the nuclei of nanoparticles. The nanodroplet concentration is determined as the number of nanodroplets per mL and the dose to be administered may be calculated over this value, however, this does not mean that the amount of PFC that shows activity is determined because the amount of PFC varies depending not only on quantity but also on size, and size distribution of nanodroplets. Moreover, these nanodroplets are the only known agents that may be used as histotripsy agents, and it is necessary to develop novel agents that are easy to prepare, that offer convenience to users, and that may serve as an alternative to said agents.

The inventors, by taking advantage of the present invention, aim to develop novel histotripsy agents that are easy to prepare, that may be attached with functional groups, that feature high stability, and that may be modified with a therapeutic agent and/or a targeting agent.

### DETAILED DESCRIPTION OF THE FIGURES

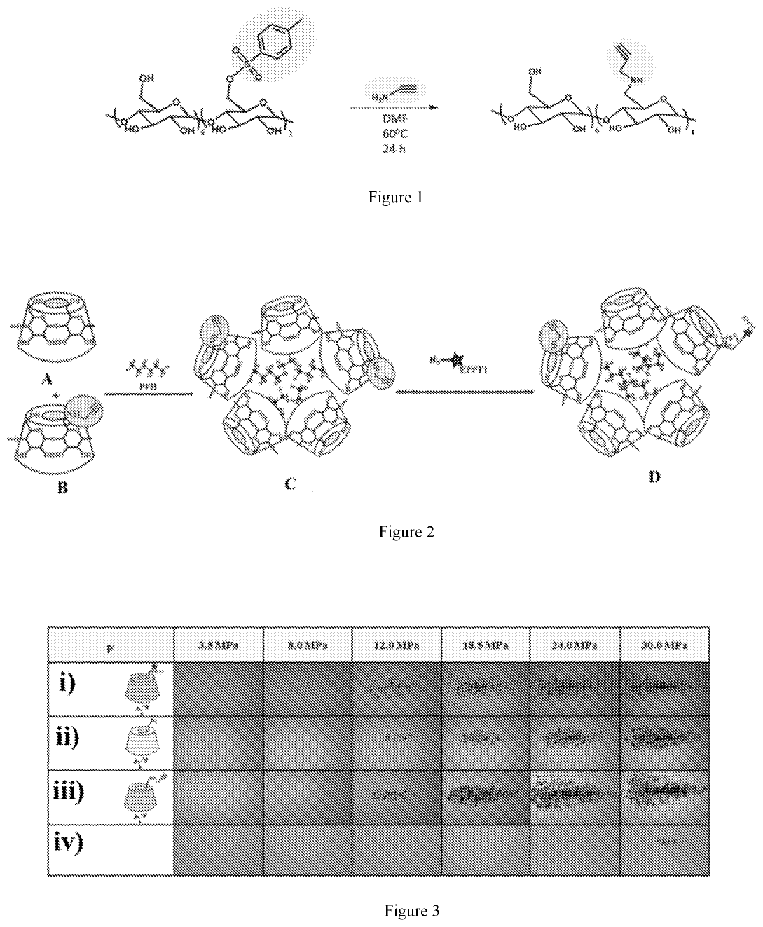

FIG. 1 illustrates the synthesis scheme of the Mono-6-Alkyne-Deoxy-6-β-Cyclodextrin molecule (Alkyne-βCD).

FIG. 2 illustrates the scheme showing the synthesis of targeted nanocone clusters and characterization thereof.

Description of the abbreviations used herein is provided below.
A: Beta-cyclodextrin
B: Beta-cyclodextrin modified with alkyne group
C: Nanocone cluster
D: Targeted nanocone cluster obtained by modifying the nanocone cluster with EPPT1 peptide.

FIG. 3 illustrates the activities of nanocontainer clusters as histotripsy agents.

Description of the abbreviations used herein is provided below.
i) Nanocone clusters modified with EPPT1
ii) Nanocone clusters containing beta-cyclodextrin modified with azide group
iii) Nanocone clusters containing beta-cyclodextrin modified with alkyne group
iv) Control

### DETAILED DESCRIPTION OF THE INVENTION

The present invention relates to nanocone clusters that are created by combining a host molecule selected from alpha-cyclodextrin, beta-cyclodextrin, gamma-cyclodextrin, a guest molecule selected from C3-C8 perfluorocarbon derivatives, and at least two inclusion complexes.

The term “alpha-cyclodextrin” used herein corresponds to a polysaccharide that is composed of six (6) glucose units bound to each other with alpha 1-4 bonds. Said molecule has a conical shape with a hydrophobic inner portion and a hydrophilic outer portion.

The term “beta-cyclodextrin” used herein corresponds to a polysaccharide that is composed of seven (7) glucose units bound to each other with a covalent bond. Said molecule has a conical shape with a hydrophobic inner portion and a hydrophilic outer portion.

The term “gamma-cyclodextrin” used herein corresponds to a polysaccharide that is composed of eight (8) glucose units bound to each other with a covalent bond. Said molecule has a conical shape with a hydrophobic inner portion and a hydrophilic outer portion.

The term “host molecule” used within the scope of the present invention corresponds to alpha-cyclodextrin or beta-cyclodextrin or gamma-cyclodextrin molecule modified with at least one of alpha-cyclodextrin or beta-cyclodextrin or gamma-cyclodextrin and/or alkyne, thiol, azide or amine groups.

The term “C3-C8 perfluorocarbon derivative” used within the scope of the present invention encompasses branched saturated fluorocarbon structures that carry C3-C8 carbon and octafluoropropane, decafluorobutane, perfluoropentane, perfluorohexane, perfluoroheptane, and perfluorooctane molecules. In a preferred embodiment of the present invention, perfluorohexane or perfluoropentane is used as a C3-C8 perfluorocarbon derivative.

The term “nanocone” used within the scope of the present invention corresponds to inclusion complex obtained when a host molecule selected from alpha-cyclodextrin, beta-cyclodextrin, gamma-cyclodextrin and a guest molecule selected from C3-C8 perfluorocarbon derivatives create a guest-host complex. In this context, the terms “guest-host complex”, “inclusion complex”, and “nanocone” used in the description provided herein have the same meaning and may be used interchangeably.

The term “nanocone clusters” used within the scope of the present invention corresponds to complexes created by the clustering of at least three nanocones. These structures are not clustered together arbitrarily, rather, the hydrophobicity of perfluorocarbons, which are the guest molecules in the inclusion complexes according to the present invention, enables clustering of these nanocones. Thus, clusters, which comprise hydrophobic perfluorocarbon chains in the inner portion and hydrophilic alpha-cyclodextrin, beta-cyclodextrin or gamma-cyclodextrin in the outer portion and inclusion complexes thereof, are created.

The present invention relates to nanocone clusters that are created through the combination of at least three inclusion complexes comprising a host molecule selected from alpha-cyclodextrin, beta-cyclodextrin, gamma cyclodextrin modified with at least on of alpha-cyclodextrin, beta-cyclodextrin, gamma-cyclodextrin and/or alkyne, thiol, azide, or amine groups and a guest molecule selected from C3-C8 perfluorocarbon derivatives.

In an embodiment of the present invention, the host molecule is composed of a mixture of alpha-cyclodextrin or beta-cyclodextrin or gamma-cyclodextrin modified with at least one of alkyne, thiol, azide, or amine groups in a rate between 1-30%, and alpha-cyclodextrin, or beta-cyclodextrin or gamma-cyclodextrin in a rate between 99% and 70%.

In this sense, the host molecule that forms the nanocone clusters according to the present invention is composed of a mixture of alpha-cyclodextrin, or beta-cyclodextrin, or gamma-cyclodextrin and alpha-cyclodextrin, or beta-cyclodextrin, or gamma-cyclodextrin modified with at least one of alkyne, thiol, azide, or amine groups. For example, the host molecule that forms the nanocone cluster according to the present invention may comprise beta-cyclodextrin and beta-cyclodextrin modified with at least one of alkyne, thiol, azide, or amine groups.

In another aspect, nanocone clusters according to the present invention may be modified with at least one therapeutic agent and/or a targeting agent.

In this aspect, the present invention relates to drug-nanocone cluster conjugates modified with at least one therapeutic agent, namely, a drug, and optionally with a targeting agent.

The term “nanocone clusters according to present invention” used in the description provided herein corresponds to structures that are obtained through the combination of at least two inclusion complexes comprising a guest molecule selected from alpha-cyclodextrin, or beta-cyclodextrin, or gamma-cyclodextrin modified with at least one of the alpha-cyclodextrin, beta-cyclodextrin, gamma-cyclodextrin and/or alkyne, thiol, azide, or amine groups and C3-C8 perfluorocarbon derivatives, and that are optionally modified with a therapeutic agent and/or a targeting agent.

The term “guest-host inclusion complex” used within the scope of the present invention corresponds to the encapsulation of a guest molecule, e.g., perfluorohexane or perfluoropentane by a host molecule, e.g., beta-cyclodextrin by means of a non-covalent interaction. In the scope of the present invention, the terms “guest-host inclusion complex”, “inclusion complex”, or “host-guest inclusion complex” or “nanocone” have the same meaning and may be used interchangeably.

Beta-cyclodextrin is a ring-shaped molecule that is composed of 7 glucose units and that has a hydrophilic outer surface and a hydrophobic inner surface. This molecule has been approved by the U.S. Food and Drug Administration (FDA) as safe to use. Hydrophobic inner surface of beta-cyclodextrin interacts and encapsulates perfluorohexane, which is a hydrophobic molecule, and the fact that the outer surface of beta-cyclodextrin has a hydrophilic structure ensures that the inclusion complex is conveyed to the targeted tissue in the hydrophilic medium of the body. The clustering occurs such that hydrophobic cyclodextrin molecules are located at the center and hydrophilic cyclodextrin molecules are located outside, as a result of the interaction between the hydrophobic free perfluorocarbon molecules, and the outer short perfluorocarbon chain piece of the structure that formed a complex with free perfluorocarbon.

The term “modified” as used herein indicates that 10% to 100%, preferably 20% to 90%, more preferably 30% to 80% of the modifiable groups on said molecule is modified with the mentioned alkyne, thiol, azide, or amine modification groups. In other words, modifiable groups on said host molecules may be available such that they are modified with the modification group at a rate of 10%, 20%, 30%, 40%, 50%, 60%, 70%, 80%, 90%, or 100%.

Perfluorohexane is a molecule that is a member of the organofluorides and has stable C—F bonds. This substance is not metabolized in the body, however, may be driven out of the body merely by inhalation. Perfluorohexane is in liquid state at ambient temperature and has a boiling temperature of 56° C. Having a low boiling point renders the use of this substance advantageous as a histotripsy agent. Thus, perfluorohexane evaporates under low pressure, thereby creating a gas cloud and forming cavitation in the tissue. Moreover, perfluorohexane is an ultrasound contrast agent. In this way, whether beta-cyclodextrin-perfluorohexane inclusion complexes reach the tumor tissue, or not, can be observed readily by means of ultrasound, and the cavitation process may be initiated once the agents reach the target tissue. Moreover, beta-cyclodextrin is stipulated to penetrate the tumor tissue better than histotripsy agents known in the state of the art and provide a more effective treatment due the fact that beta-cyclodextrin has a small size and a uniform structure.

The term “targeting agent” used herein corresponds to molecules that have the tendency to bind to various specific target tissues in the body. In other words, targeting agents correspond to molecules that have a tendency to bind to cells with special receptors specific thereto.

Targeting agents that may be used within the scope of the present invention may be selected from folic acid, antibodies, antibody fragments, or various peptides. In a preferred embodiment of the present invention, EPPT1 peptide is used as a targeting agent.

An embodiment of the present invention relates to a method employed for the preparation of nanocone clusters according to the present invention, wherein said method comprises the process steps of;
a. Dissolving the host molecule, e.g., beta-cyclodextrin in water,
b. Adding a guest molecule that is selected from C3-C8 perfluorocarbon derivatives, for example perfluorohexane, to the solution in an amount, in moles, of 1-10 times, preferably 3-6 times, more preferably 5 times of the amount of the host molecule; used
c. Separating the solid and liquid portions of the obtained precipitation, drying the solid portion, thereby obtaining nanocone clusters.

In an embodiment of the present invention, beta-cyclodextrin solution mixture may be heated to a certain temperature in order to ensure dissolving in step (a), wherein said solution is heated preferably to 70-90° C., and more preferably to 80° C.

In the method according to the present invention, using C3-C8 perfluorocarbon derivative, for example, perfluorohexane or perfluorocarbon having an equivalence of 1-10 moles, preferably 3-6 moles, more preferably 5 moles to alpha-cyclodextrin, or beta-cyclodextrin, or gamma-cyclodextrin modified with at least one of alpha-cyclodextrin, beta-cyclodextrin, gamma-cyclodextrin and/or alkyne, thiol, azide, or amine groups in the host molecule having an equivalence of 1 mole assumes a crucial role in the formation of nanocone clusters.

In a preferred embodiment of the present invention, a method used for the preparation of nanocone clusters comprises the process steps of;
a. Dissolving beta-cyclodextrin and beta-cyclodextrin modified with at least one of alkyne, thiol, azide, or amine groups in a suitable solvent,
b. Adding perfluorohexane or perfluoropentane to the solution at an amount 5 times of the total mole amount of beta-cyclodextrin and beta-cyclodextrin modified with at least one of alkyne, thiol, azide, or amine groups,
c. Separating the solid and liquid portions of the obtained precipitation, drying the solid portion thereof, and obtaining beta-cyclodextrin/amine or alkyne or azide or thiol beta-cyclodextrin perfluorohexane or perfluoropentane nanocone clusters.

In another aspect, the present invention relates to nanocone clusters according to the present invention suitable for use as a histotripsy agent or an ultrasound contrast agent.

In another aspect, the present invention relates to the use of nanocone clusters according to the present invention as a histotripsy agent or an ultrasound contrast agent.

In another aspect, the present invention relates to nanocone clusters according to the present invention for use in the treatment of cancer.

In another aspect, the present invention relates to drug-nanocone cluster conjugates according to the present invention for use in the treatment of cancer.

The term “cancer” used herein corresponds to a physiological condition characterized by malignant tumors or uncontrolled cell growth. Examples of cancer may include but are not limited to carcinoma, lymphoma, blastoma, sarcoma, and leukemia.

Carcinoma, as used herein, corresponds to a type of cancer that consists of epithelial cells.

Lymphoma, as used herein, corresponds to a type of cancer that develops from lymphocytes.

Blastoma, as used herein, corresponds to a type of cancer that develops from precursor cells also known as blast cells.

Sarcoma, as used herein, corresponds to a type of cancer stemming from transformed cells of mesenchymal origin.

Leukemia, as used herein, corresponds to a type of cancer that begins in the bone marrow and results in high counts of abnormal white blood cells.

More specific examples of cancer types include; breast cancer, prostate cancer, colorectal cancer, skin cancer, small cell lung cancer, non-small cell lung cancer, mesothelioma, gastrointestinal cancer, pancreatic cancer, glioblastoma, vulva cancer, cervical cancer, endometrial carcinoma, ovarian cancer, liver cancer, hepatoma, bladder cancer, kidney cancer, salivary gland carcinoma, thyroid cancer, and various head and neck cancers.

In another aspect, the present invention relates to pharmaceutical compositions comprising nanocone clusters or drug-nanocone conjugates according to the present invention.

In a preferred embodiment of the present invention, nanocone clusters contained in the pharmaceutical composition consist of beta-cyclodextrin or beta-cyclodextrin functionalized with alkyne, thiol, or amine group and perfluorohexane or perfluoropentane.

The present invention further discloses pharmaceutical compositions comprising beta-cyclodextrin and perfluorohexane or perfluoropentane nanocone clusters.

The present invention further describes pharmaceutical compositions comprising at least one therapeutic agent, preferably an antineoplastic agent conjugated with beta-cyclodextrin and perfluorohexane or perfluoropentane nanocone clusters.

Antineoplastic agent mentioned within the scope of the present invention may be selected from small-molecule antineoplastic agents or antineoplastic agents of biological origin. Antineoplastic agents of biological origin may be, for instance, proteins, peptides, antibodies, and monoclonal antibodies.

The mentioned antineoplastic therapeutic agents may assume any role in the treatment of cancer. Said agents, for example, may be any agent that produces effects such as cell cycle arrest, inhibition of vascular endothelial growth factor (VEGF), altering immune response, etc.

In a preferred embodiment of the present invention, pharmaceutical compositions containing nanocone clusters or drug-nanocone conjugates comprise at least one excipient in addition to the inclusion complex according to the present invention.

Said excipient is an agent used to meet the criteria such as solubility, dispersion, dose uniformity, etc., of the pharmaceutical composition according to the present invention and does not produce any pharmaceutical activity.

Pharmaceutical compositions comprising inclusion complexes according to the present invention may be formulated in any dosage form available in the state of the art. In a preferred embodiment of the present invention, dosage forms according to the present invention are in an injectable form. These injectable forms may be prepared to be administered preferably via intravenous, intraperitoneal, intratracheal administration routes.

The present invention will now be described by making references to examples given below that are provided only for illustrative purposes and should not be construed to impose any limiting effects on the scope of the present invention.

### EXAMPLES

### Example 1: Preparation of Beta-Cyclodextrin Perfluorohexane Inclusion Complex

100 mg of beta-cyclodextrin is mixed with 6 mL of water. Perfluorohexane is added in 5-fold molar ratios once the beta-cyclodextrin is dissolved completely. The mixture is stirred overnight at ambient temperature. Then, having been kept at 4° C. for an hour in a refrigerator, the mixture is centrifuged at 5000 rpm for 10 minutes (12000 rpm for 2 minutes). The liquid portion is removed and obtained solids are dried under vacuum.

### Example 2: Synthesis of Mono-6-Alkyne-Deoxy6-β-Cyclodextrin (Alkyne-βCD)

For Alkyne-PCD synthesis, Ts-CD (1 eq) was dissolved inside dimethylformamide (DMF) under nitrogen atmosphere and propargylamine (38 eq) was added thereto. The reaction was heated to 80° C. by using an oil bath and stirred overnight. The product was dissolved in MeOH/water mixture several times, precipitated in acetonitrile and was obtained as a white solid. The obtained solid was dried under vacuum (FIG. 1).

### Example 3: Synthesis of Mono-6-Azido-Deoxy-6-Cyclodextrin (Azide-βCD)

For the synthesis of Azide-βCD, Ts-CD (1 eq) and NaN3 (20 eq) was mixed overnight in 300 mL of water at 1000 C in the presence of a condenser. After the mixture was filtered, the volume of the mixture was reduced to approximately 15 mL by means of vacuum distillation. 1,1,2,2-tetrachloroethane (15 eq) was added thereto and stirred for half an hour. Following centrifugation at 4° C. 3500 rpm for 15 minutes, the solid phase was removed and purified by means of crystallization. Then, the pure solid was dried under vacuum.

### Example 4: Synthesis of Mono-6-Amino-Deoxy-6-Cyclodextrin (Amine-βCD)

Azide-BCD (1 eq) and triphenylphosphine (1.1 eq) were mixed in an inert medium for 2 hours at ambient temperature. Some water was added thereon, and the mixture was left for stirring once again at 90° C. for 3 hours in a spiral cooler. At the end of this period, the cooled mixture was reduced to ⅓ of the initial volume by means of vacuum distillation and precipitated by adding acetone. Following the centrifugation at 4° C. and 3500 rpm for 5 minutes, the solid phase was collected and dried by means of vacuum.

### Example 5: Preparation of Functional Nanocone Clusters (Non-Targeted Nanocone Clusters)

Functional nanocone clusters were prepared by using 20% functional cyclodextrin (CD) derivative (Azide-CD or Alkyne-CD or Amine-CD) by mass through mixing with 80% beta-cyclodextrin (BCD) and adding 5-fold PFH by mole. This ensures that all components are initially in a soluble state. Obtained functional nanocone clusters were used both in PEG inoculation and EPPT1 peptide inoculation (targeted nanocone cluster).

For nanocone clusters with alkyne function, alkyne-BCD (1 eq) and BCD (4 eq) were dissolved inside 5 mL of double distilled water at 700 rpm by using a magnetic stirrer. After a clear solution was obtained, PFH (20 eq) was added, and the solution was kept in a refrigerator at 4° C. overnight following a process of stirring overnight. Then, the solution was centrifuged for 15 minutes at 5000 rpm and the final precipitation was dried under vacuum.

### Example 6: Synthesis and Characterization of Targeted Nanocone Clusters

Having been selected as a targeting agent, EPPT1 had been pre-synthesized and purchased in a manner to contain peptide azide end-group. Therefore, functional nanocone clusters comprising alkyne groups may be functionalized with this peptide.

Alkyne-BCD nanocone clusters were dispersed inside 1.9 mL of double distilled water at ambient temperature and at 750 rpm by using a magnetic stirrer. N₃-EPPT1 (0.05 eq) and CuSO₄ (1 eq) and sodium ascorbate (2 eq) were added, and the system was left under a nitrogen atmosphere. The reaction was stirred for 24 hours at ambient temperature in a dark environment. After the “Click” reaction was complete, the CuSO₄/sodium ascorbate system was washed with water and removed by means of centrifugation (FIG. 2).

### Example 7: Examination of Histotripsy Activities of Nanocone Clusters

The cavitation threshold pressures of stable complexes obtained thus far were tested with nanocone clusters placed inside a previously-deaerated agarose gel. The same amount of nanocone cluster was used in order to determine the cavitation threshold pressure since these structures were prepared in the same concentration and in the same PFH ratio. In the conducted study, a comparison was made by equalizing the amount of nanocone per mL. Subsequent to this stage, a 500-kHz transducer was employed with the purpose of applying single-loop histotripsy pulses and the pulse repetition frequency was adjusted to 1 Hz. Results were recorded by using a high-speed camera. As can be seen in FIG. 3, targeted nanocones and functional nanocones were subjected to a comparison with normal BCD nanocone clusters in normal histotripsy. Pressure drop was observed in all targeted and non-targeted nanocone clusters.

1. Nanocone clusters that comprise a host molecule selected from alpha-cyclodextrin, beta-cyclodextrin, gamma-cyclodextrin modified with at least one of alpha-cyclodextrin, beta-cyclodextrin, gamma-cyclodextrin and/or alkyne, thiol, azide or amine groups, and a guest molecule selected from C3-C8 perfluorocarbon derivatives, and that are created through the combination of at least two inclusion complexes.

2. Nanocone clusters according to claim 1, wherein the C3-C8 perfluorocarbon derivative is selected from branched saturated fluorocarbon structures that carry C3-C8 carbon and octafluoropropane, decafluorobutane, perfluoropentane, perfluorohexane, perfluoroheptane, and perfluorooctane molecules.

3. An inclusion complex according to claim 1, wherein the guest molecule is perfluorohexane or perfluoropentane.

4. Nanocone clusters according to claim 1, wherein they are formed by the clustering of at least three nanocones.

5. Nanocone clusters according to claim 1, wherein the host molecule is composed of a mixture of alpha-cyclodextrin, or beta-cyclodextrin, or gamma-cyclodextrin and alpha-cyclodextrin, or beta-cyclodextrin, or gamma-cyclodextrin modified with at least one of alkyne, thiol, azide, or amine groups.

6. Nanocone clusters according to claim 5, wherein the host molecule is composed of a mixture of alpha-cyclodextrin or beta-cyclodextrin or gamma-cyclodextrin modified with at least one of alkyne, thiol, azide, or amine groups in a rate between 1-30%, and alpha-cyclodextrin, or beta-cyclodextrin or gamma-cyclodextrin in a rate between 99% and 70%.

7. Nanocone clusters according to claim 5, wherein the host molecule is composed of beta-cyclodextrin modified with at least one of beta-cyclodextrin and alkyne, thiol, or amine groups.

8. Nanocone clusters according to claim 1, wherein they are functionalized with at least one therapeutic agent and/or targeting agent.

9. Nanocone clusters according to claim 8, wherein the targeting agent is selected from folic acid, antibodies, antibody fragments or various peptides.

10. Nanocone clusters according to claim 7, wherein the therapeutic agent is an antineoplastic agent, preferably small-molecule antineoplastic agent, or antineoplastic agents of biological origin.

11. Drug-nanocone cluster conjugates obtained by modifying the nanocone cluster according to claim 1 with at least one therapeutic agent.

12. Drug-nanocone cluster conjugates according to claim 11, wherein the nanocone clusters are modified with a targeting agent.

13. Pharmaceutical compositions comprising drug-nanocone cluster conjugates according to claim 11 as active substance.

14. Pharmaceutical composition according to claim 13, comprising at least one excipient.

15. A method for use in the preparation of nanocone clusters according to claim 1, comprising the process steps of:
a. dissolving the host molecule in a suitable solvent,
b. adding a guest molecule that is selected from C3-C8 perfluorocarbon derivatives to the solution in an amount, in moles, of 1-10 times of the amount of the host molecule used, and
c. separating the solid and liquid portions of the obtained precipitation, drying the solid portion, thereby obtaining nanocone clusters.

16. A method for use in the preparation of nanocone clusters according to claim 15, comprising the process steps of:
a. dissolving beta-cyclodextrin and beta-cyclodextrin modified with at least one of alkyne, thiol, azide, or amine groups in a suitable solvent,
b. adding perfluorohexane or perfluoropentane to the solution in an amount 5 times of the total mole amount of beta-cyclodextrin and beta-cyclodextrin modified with at least one of alkyne, thiol, azide, or amine groups, and
c. separating the solid and liquid portions of the obtained precipitation, drying the solid portion thereof, and obtaining beta-cyclodextrin/amine or alkyne or azide or thiol beta-cyclodextrin perfluorohexane or perfluoropentane nanocontainer clusters.

17. A method according to claim 15, wherein the solvent used in step (a) is selected from an organic solvent, or water, or any aqueous solution.

18. Nanocone clusters according to claim 1, for use as histotripsy agent or ultrasound contrast agent.

19. Nanocone clusters according to claim 1, for use in the treatment of cancer.

20. Drug-nanocone cluster conjugates according to claim 11, for use as histotripsy agent or ultrasound contrast agent.

21. Drug-nanocone cluster conjugates according to claim 11, for use in the treatment of cancer.

22. Pharmaceutical compositions according to claim 13 for use in the treatment of cancer.
